(19) United States
(12) Patent Application Publication
(10) Pub. No.: US 2009/0130048 A1
(43) Pub. Date: May 21, 2009

(54) Topical Composition for Treating Pain

(76) Inventors: Bryan Todd Oronsky, Los Altos Hills, CA (US); Neil Charles Oronsky, Los Altos Hills, CA (US); Arnold L. Oronsky, Los Altos Hills, CA (US)

Correspondence Address: MORRISON & FOERSTER LLP, 755 PAGE MILL RD, PALO ALTO, CA, 94304-1018, US

(21) Appl. No.: 11/942,409

(22) Filed: Nov. 19, 2007

(51) Int. Cl. A61K 31/765; A61K 31/75; A61P 29/00

(52) U.S. Cl. 424/78.02

(57) ABSTRACT

Topical compositions having as the active ingredient a hydrophilic material, such as a polyalkylene oxide homopolymer or copolymer, and methods of use, have been developed for the amelioration or prevention of pain or the sequelae of pain. The composition may be in the form of a cream, gel, lotion, spray, foam, paste, patch, suspension or dispersion. In the preferred embodiment, the formulation is a gel. The composition may contain a penetration enhancer, most preferably one with membrane disruptive properties. In one embodiment, the compositions are incorporated onto or into disposables such as hemorrhoid wipes, gauze, sponge, bandages, and wraps; mouth guards, dental trays; needles or catheters; adult diapers; gloves, socks or wrist bands, for ease of application. The composition is applied topically to a site at or adjacent to a painful region. The composition is reapplied as necessary. Pain relief is typically obtained within minutes and lasts for periods of variable duration ranging from minutes to several hours and even, in some cases, days. The composition is variably effective to treat visceral, somatic and neuropathic pain both acute and chronic as well as muscle pain and stiffness and joint pain and stiffness.

### FIELD OF THE INVENTION

The present invention relates to a topical treatment of acute and chronic pain, which is can be somatic, visceral, or neuropathic, as well as joint and muscle stiffness. This treatment also addresses to some degree the psychological, vegetative and medication-induced sequelae of pain (usually chronic) which can include fatigue, decreased alertness, weight gain, decreased exercise tolerance, and dyspnea.

### BACKGROUND OF THE INVENTION

Pain is a sensation and a perception that is comprised of a complex series of mechanisms. In its most simple construction, it is a signal from the firing of nociceptors, touch and pressure receptors in the periphery, that is transmitted to the spinal cord and finally to lower and higher centers of the brain. However, this signal can be modified in a multitude of ways at each level of the pain pathway. See e.g. Millan, M. J. (1999) The Induction of Pain: An Integrative Review, Progress in Neurobiology, 57, 1-164 (Pergamon Press) for an in depth review.

There are primarily three types of pain: somatic, visceral and neuropathic, all of which can be acute and chronic. Somatic pain is caused by the activation of pain receptors in either the cutaneous or musculoskeletal tissues. In contrast to surface somatic pain which is usually described as sharp and may have a burning or pricking quality, deep somatic pain is usually characterized as a dull, aching but localized sensation. Somatic pain may include fractures in the vertebrae, joint pain (deep somatic pain) and postsurgical pain from a surgical incision (surface pain).

Visceral pain is caused by activation of pain receptors in internal areas of the body that are enclosed within a cavity. Visceral pain is usually described as pressure-like, poorly localized and deep.

Neuropathic pain, caused by neural damage, is usually described as burning, tingling, shooting or stinging but can also manifest itself as sensory loss either as a result of compression, infiltration, chemical or metabolic damage or is idiopathic. Examples of neuropathic pain are heterogenous and include medication-induced neuropathy and nerve compression syndromes such as carpal tunnel, radiculopathy due to vertebral disk herniation, post-amputation syndromes such as stump pain and phantom limb pain, metabolic disease such as diabetic neuropathy, neurotropic viral disease from herpes zoster and human immunodeficiency virus (HIV) disease, tumor infiltration leading to irritation or compression of nervous tissue, radiation neuritis, as after cancer radiotherapy, and autonomic dysfunction from complex regional pain syndrome (CRPS).

Inflammatory pain is related to tissue damage which can occur in the form of penetration wounds, burns, extreme cold, fractures, inflammatory arthropathies as seen in many autoimmune conditions, excessive stretching, infections, vasoconstriction and cancer.

Acute pain, termed nociception, is the instantaneous onset of a painful sensation in response to a noxious stimulus. It is considered to be adaptive because it can prevent an organism from damaging itself. For example, removing a hand from a hot stove as soon as pain is felt can prevent serious burns. The second type of pain is persistent pain. Unlike acute pain, it usually has a delayed onset but can last for hours to days. It is predominately considered adaptive because the occurrence of persistent pain following injury can prevent further damage to the tissue. For example, the pain associated with a sprained ankle will prevent the patient from using the foot, thereby preventing further trauma and aiding healing, A third category of pain is chronic pain. It has a delayed onset and can last for months to years. In contrast to acute and persistent pain, chronic pain is considered maladaptive and is associated with conditions such as arthritis, nerve injury, AIDS and diabetes. Yet another type of pain can be termed breakthrough pain. This is a brief flare-up of severe pain lasting from minutes to hours that can occur in the presence or absence of a preceding or precipitating factor even while the patient is regularly taking pain medication. Many patients experience a number of episodes of breakthrough pain each day.

Many types of pain control are systemic in nature. These controls, however, also have systemic side effects, such as stomach ulcers (e.g., non-steroidal antiinflammatories (“NSAIDS”)), hepatotoxicity (e.g., acetaminophen), constipation, CNS effects, respiratory depression, drug tolerance, dependence, and addiction from opioid narcotics and impotence and decreased libido from antidepressants. In the case of chronic pain, the side effects from these systemic medications sometimes can be controlled only with the addition of more systemic medications which in turn have their own side effects such as the psychostimulant Ritalin to help counteract the symptoms of opioid-related drowsiness. In addition, the psychological component of chronic pain can lead to fatigue, weight gain, increased appetite, decreased concentration and awareness, decreased energy, and psychomotor retardation, which often require further adjunctive therapy such as antidepressants and stimulants. Associated comorbid conditions such as COPD, asthma, and hypertension can lead to dyspnea and decreased exercise tolerance, thereby exacerbating the downward spiral and depression which often characterizes chronic pain syndromes and necessitate further adjunctive treatment. Moreover, most topical treatments to control pain, such as lidocaine sprays and patches and benzocaine ointments, are of limited efficacy and/or last only for a few minutes.

Pain of all types can be debilitating, both psychologically and physically, and exacts an enormous toll in dollars, decreased productivity, and quality of life Therefore, formulations for prevention or alleviation of pain that are effective, safe, allow for increased levels of patient control, and in some measure affect the important psychological, vegetative and medication-related sequelae of pain symptoms and treatment are needed in order to increase functionality and decrease the use of systemic medications with their attendant side effects.

It is therefore an object of the present invention to provide topical formulations providing pain relief for periods of varying durations lasting from minutes, to hours to days depending on the patient and the type of pain and painful lesion or syndrome.

### SUMMARY OF THE INVENTION

Topical compositions having as the active ingredient a hydrophilic material, such as polyalkylene oxide homopolymer or copolymer, and methods of use, have been developed for the amelioration or prevention of pain or the sequelae of pain. The composition may be in the form of a cream, gel, lotion, spray, foam, paste, patch, suspension, dispersion, or pad for use with a needle stick, such as a diabetic needle stick or lancet, In the preferred embodiment, the formulation is a gel. The composition may contain a penetration enhancer, most preferably one with membrane disruptive properties. In one embodiment, the compositions may also include one or more additional active ingredients. In another embodiment, the compositions are incorporated onto or into disposables such as hemorrhoid wipes, gauze, sponge, bandages, and wraps; mouth guards, dental trays; needles, needle sticks or catheters; adult diapers; gloves, socks or wrist bands, for ease of application.

The composition is applied topically to a site at or adjacent to a painful region. The composition is reapplied as necessary. Pain relief is typically obtained within minutes and lasts for periods of variable duration ranging from minutes to several hours and even, in some cases, days. The compounds are applied such that the dosage is sufficient to provide an effective dose in the painful area or immediately adjacent areas, to ameliorate or eliminate pain. The composition is variably effective to treat visceral, somatic and neuropathic pain both acute and chronic as well as muscle pain and stiffness and joint pain and stiffness. Examples demonstrate pain relief in human patients for a wide number of conditions, including joint, muscle and tendon pain, joint, muscle and tendon immobility, inflammatory pain, neuropathies, muscle spasms, osteoarthritis, breathing disorders such as wheezing, hunger pains, some types of headaches, dysphagia, fibromyalgia, autoimmune disorders, and pancreatitis.

### DETAILED DESCRIPTION OF THE INVENTION

### I. Definitions

“Water Soluble” as used herein refers to substances that have a solubility of greater than or equal to 5 g/100 ml water.

“Lipid Soluble” as used herein refers to substances that have a solubility of greater than or equal to 5 g/100 ml in a hydrophobic liquid such as castor oil.

“Hydrophilic” as used herein refers to substances that have strongly polar groups that readily interact with water.

“Lipophilic” refers to compounds having an affinity for lipids.

“Amphiphilic” refers to a molecule combining hydrophilic and lipophilic (hydrophobic) properties “Hydrophobic” as used herein refers to substances that lack an affinity for water; tending to repel and not absorb water as well as not dissolve in or mix with water.

An “oil” is a composition containing at least 95% wt of a lipophilic substance. Example lipophilic substances include but are not limited to naturally occurring and synthetic oils, fats, fatty acids, lecithins, triglycerides and combinations thereof.

An “emulsion” is a composition containing a mixture of non-miscible components homogenously blended together. In particular embodiments, the non-miscible components include a lipophilic component and an aqueous component. An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4^{th} Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is an emulsion having a viscosity of between 100 and 1000 centistokes.

A “cream” is an emulsion having a viscosity of greater than 1000 centistokes, typically in the range of 20,000-50,000 centistokes.

A “paste” is a liquid or emulsion having solid material homogenously suspended therein, typically in a lotion cream or gel.

A “gel” is a composition containing a thickening agent or polymeric material dissolved or suspended in a liquid. The liquid may include a lipophilic component an aqueous component or both. Some emulsions may be gels or otherwise include a gel component. Some gels, however, are not emulsions because some do not contain a homogenized blend of immiscible components.

“Penetration enhancers” are used to promote transdermal delivery of drugs across the skin, in particular across the stratum corneum. These can be chemical penetration enhancers or physical penetration enhancers, such as ultrasound.

Skin protectants can be included in compositions formulated for topical administration. Such agents not only soothe the site of infection but may also aide in maintaining the integrity of the skin to prevent additional damage. Suitable skin protectants include allantoin; cocoa butter; dimethicone; kaolin; shark liver oil; petrolatum; lanolin; vegetable oils; ethoxylated oils and lipids; polymers such as polyalkylene oxides, polyvinylpyrrolidone, polyvinyl alcohol, poly(meth)acrylates, ethylvinyl acetate, polyalkylene glycols; polysaccharides and modified polysaccharides such as hyaluronic acid, cellulose ethers, cellulose esters, hydroxypropyl methylcellulose, crosscarmelose, and starch; natural gums and resins which may be gelling or non-gelling such as alginates, carrageenans, agars, pectins, glucomannans (guar, locust bean, etc.), galactomannans (e.g. konjac), gum arabic, gum traganth, xanthan, schleroglucan and shellac; and colloidal insolubles such as zinc oxide and other insoluble zinc salts, talcum powder and other micronized natural minerals; and colloidal silicas, aluminas and other metal oxides.

Buffers are used to control pH of a composition. Preferably, the buffers buffer the composition from a pH of about 4 to a pH of about 7.5, more preferably from a pH of about 4 to a pH of about 7, and most preferably from a pH of about 5 to a pH of about 7. In a preferred embodiment, the buffer is triethanolamine.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, and thimerosal.

### II. Compositions

As demonstrated by the examples, it has been discovered that certain hydrophilic materials, alone or in combination with a lipophilic vehicle (LV), can alleviate or prevent pain from a variety of different sources, when applied topically. Although the vehicles can also be used for drug delivery, drug is not required for efficacy.

A. Polyalkylene Oxides and Alklene Oxides

The active ingredient can be a hydrophilic polymer, such as a polyalkyleneoxide or derivatives thereof. Topical compositions having as the active ingredient a polyalkylene oxide homopolymer, copolymer, or combinations thereof have been developed and tested. Other hydrophilic materials, such as propylene oxide, may also be used. The formulation typically includes excipients that are used to form a cream, gel, lotion, spray, foam, paste, patch or pad, suspension or dispersion, for topical application to the skin or mucosal surface.

Suitablepolyalkylene oxides include, but are not limited to, polyethylene glycol (“PEG”, also referred to as polyethylene oxide “PEO”), polypropylene oxide (“PPO”), polyethylene oxide-co-propylene oxide (“PEO-PPO”)copolymers(available under the trade name Pluronics®), derivatives of polyalkylene oxides, such as mono or diesters of a polyalkylene oxides (e.g., polyoxyl 40 stearate), and combinations thereof

1. Polyethylene Glycol

PEG is prepared by the polymerization of ethylene oxide. PEG is typically a liquid or low-melting solid at room temperature depending on the molecular weight of the polymer. Poly (ethylene glycol) is produced by interaction of calculated amount of ethylene oxide with water, ethylene glycol or ethylene glycol oligomers. The reaction can be catalyzed by acidic or basic catalysts. Depending on the catalyst type the mechanism of polymerization can be cationic or anionic. Anionic polymerization is more preferable because it allows one to obtain PEG with low polydispersity. Polyethylene oxide or high-molecular polyethylene glycol can be synthesized via suspension polymerization.

2. Pluronics

Pluronics®, also known as poloxamers, are block copolymers containing ethylene oxide and propylene oxide. Pluronics® have been used as antifoaming agents, wetting agents, dispersants, thickeners, and emulsifiers. Because of their amphiphilic structure, poloxamers have surfactant properties that make them useful in industrial applications. Among other things, they can be used to increase the water solubility of hydrophobic, oily substances or otherwise increase the miscibility of two substances with different hydrophobicities. For this reason, these polymers are commonly used in industrial applications, cosmetics, and pharmaceuticals. They have also been used as model systems for drug delivery applications.

Pluronic® F-127 is a polaxamer surfactant which is an ABA-type block copolymer containing 70% polyethylene oxide (PEO). The molecular weight is 12,500 Daltons. Upon cooling, Pluronic® F-127 becomes a liquid, while at higher temperatures, the material is a solid or semi-solid. DMSO and lecithin/isopropyl palmitate can be added to Pluronic® F-127 to increase absorption through the skin.

3. Propylene Oxide

Propylene oxide formulations can also be used. For example, SURGILUBE™ contains the following ingredients: water, propylene oxide, chlorhexidine gluconate 20%, acetic acid, lavender, hydroxypropyl methylcellulose, polypropylene glycol, Sodium Acetate, Propylene Glycol. Surgilube is a medical lubricant used to coat catheters and other medical equipment and is also placed on gloves for rectal and vaginal exams. Chlorhexidine gluconate is an antiseptic and preservative. Note, however, that SURGILUBE™ is not as effective as other formulations described herein, possibly due to lower concentration of polymer.

B. Penetration Enhancers

In a preferred embodiment, the composition penetrates into the skin. The composition may contain a penetration enhancer, most preferably one with membrane disruptive properties. One long-standing approach for improving transdermal drug delivery uses penetration enhancers (also called sorption promoters or accelerants) which penetrate into skin to reversibly decrease the barrier resistance. Numerous compounds have been evaluated for penetration enhancing activity, including sulphoxides (e.g., dimethylsulfoxide (“DMSO”) and decylmethylsulfoxide (C10MSO)), Azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes. Many potential sites and modes of action have been identified for skin penetration enhancers, such as the intercellular lipid matrix in which the accelerants may disrupt the packing motif, the intracellular keratin domains, or through increasing drug partitioning into the tissue by acting as a solvent for the permeant within the membrane. Further potential mechanisms of action, for example with the enhancers acting on desmosomal connections between corneocytes or altering metabolic activity within the skin, or exerting an influence on the thermodynamic activity/solubility of the drug in its vehicle are possible.

Preferred penetration enhancers include the sulfoxide decylmethylsulfoxide (C10MSO); ethers such as diethylene glycol monoethyl ether, dekaoxyethylene-oleylether, and diethylene glycol monomethyl ethers; surfactants, fatty acids such as C₈-C₂₂ and other fatty acids, C₈-C₂₂ fatty alcohols, and polyols. Other suitable penetration enhancers include, but are not limited to, urea, (carbonyldiamide), imidurea, N,N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as Brij® (76 (stearyl poly( 10 oxyethylene ether), Brij® 78 (stearyl poly(20)oxyethylene ether), Brij® 96 (oleyl poly(10)oxyethylene ether), and Brij® 721 (stearyl poly (21) oxyethylene ether) (ICI Americas Inc. Corp.). Fatty acids such as linoleic acid, capric acid, lauric acid, and neodecanoic acid, which can be in a solvent such as ethanol or propylene glycol, can be used as lipid bilayer disrupting agents. DMSO is not a particularly preferred penetration enhancer due to its strong odor and the fact that it is not approved for use in humans by the Food and Drug Administration.

Detergents such as Dawn® detergent contain sodium lauryl sulfate, sodium pareth-23. Sodium dodecyl sulfate (or sulphate) (SDS or NaDS) (C₁₂H₂₅NaO₄S), also known as sodium lauryl sulfate (SLS), is an ionic surfactant that is used in household products such as toothpastes, shampoos, shaving foams and bubble baths for its thickening effect and its ability to create a lather. The molecule has a tail of 12 carbon atoms, attached to a sulfate group, giving the molecule the amphiphilic properties required of a detergent.

C. Formulations and Kits

The hydrophilic polymer composition can be administered directly or used in combination with a composition, device or formulation. For example, the hydrophilic polymer composition can be impregnated onto or into bandages or adhesive strips such as Band-Aids®. These will then alleviate pain, prevent sticking to the wound, and allow the absorbent material to absorb liquid and protect the injury. The hydrophilic compositions can be impregnated in combination with an antiseptic. Common antiseptics and preservatives include ethanol, 1-propanol, and 2-propanol/isopropanol, benzalkonium chloride (BAC), cetyl trimethylammonium bromide (CTMB), cetylpyridinium chloride (Cetrim), cetylpyridinium chloride (CPC) and benzethonium chloride (BZT), boric acid, chlorhexidine gluconate, iodine, mercurochrome, octenidine dihydrochloride, and phenol compounds.

The hydrophilic polymer composition s can be administered in combination with gauze, sponge, cotton swab (one or two sided or ended), wrap, patch, dressing, medication pad, tissue, pain-relief gel pack, lip balm, poultice, plaster, or compress.

The hydrophilic polymer composition can be applied within, on or in devices such as gloves, socks, wrist bands. The hydrophilic polymer can be impregnated into a wipe for use in alleviating pain from hemorrhoids or anal fissures. The gloves, socks or wristbands may have the formulation applied to the inside as a coating, impregnated into the fibers, or provided as a separate applicator for administration at the time of application. They may be applied as built-in or attach-on disposable pads to mattresses and pillows such as cervical pillows. The hydrophilic polymer composition may be applied to cushioned insoles and corn and bunion pads to help alleviate pain in the feet. The hydrophilic polymer composition may be applied on, in or to compression stockings such as TED hose or Jobst stockings to alleviate the pain of varicose veins and superficial thrombophlebitis.

The hydrophilic polymer composition may be used in facial tissues to soothe or prevent the sore or chapped skin under or around the nose with allergies or upper respiratory infections.

They may be used to coat medical instruments to ease the pain of their insertion and simultaneously to provide lubrication such as with a catheter.

They may be used to coat metal-containing items such as jewelry, hooks, zippers, pens, snaps and tools for individuals who have metal allergies and in particular nickel sensitivity.

They may be applied to mechanical braces, sleeves, corsets and girdles, splints, casts, prostheses and the like to provide analgesia along with the functional and positional support provided by the orthoses.

They may be applied as built-in or attach-on disposable pads to superficial heating devices such as electric heating pads, rubber hot water bottles, warm fluid heat packs, chemical hot packs and therapeutic cold modalities such as ice packs or added to vapocoolant sprays. They may be used in concert with modalities of electrotherapy such as iontophoresis, TENS, muscle stimulation, and diathermy or applied to the electrodes of these devices. They may be used in concert with radiation therapy such as infrared, ultraviolet and cold laser.

Examples of disposables include patches, hemorrhoid wipes, medication pads, dressings, gauze, sponges, bandages, tissues, wraps, pain-relief gel packs and beds, swab sticks and Q-tips, poultices, plasters and compresses; devices and equipment for injury protection, increased mobility, functional and positional support and correction such as orthotics, braces, TED hose and other support stockings, crutches, casts, splints, prosthetics, girdles and corsets, hot water bottles, inserts, insoles and arch supports, pads (e.g. corn and bunion) exercise equipment, cooling or heating devices, mattresses, pillows, chucks and bed liners and mouth guards; medical, dental and surgical implants, equipment and supplies such as dental trays, dental bridges, dentures, crowns, floss, picks, needles, lancets, rods, stents, blades, probes, stylets, tubes, scissors, clamps, retractors, forceps, endoscopes, mammography compression plates, cannulas or catheters; articles of clothing and footwear including shoes, shoelaces, socks, gloves, caps, scarves, leotards, head bands, wrist bands, gloves and adult diapers, pads, guards and liners. Additional materials include patches, pads, bandages or dressings for use around the neck to decrease obstructive sleep apnea.

The hydrophilic polymer composition may be applied as built-in or attach-on disposable pads to mattresses and pillows such as cervical pillow. The hydrophilic polymer composition may be applied to bed underpads and chucks to alleviate the pain of bed sores and to promote continence. The hydrophilic polymer composition may be applied to cushioned insoles and corn and bunion pads to help alleviate pain in the feet. The hydrophilic polymer composition may be applied on, in or to compression stockings such as TED hose or Jobst stockings to alleviate the pain of varicose veins and superficial thrombophlebitis.

The hydrophilic polymer composition may be applied to one end of a two-sided swab stick. The other end of the swab stick could contain a disinfectant like alcohol or iodine or an antihistamine or anti-inflammatory as well as antibiotics, chemotherapeutic agents, minerals and vitamins, appetite-suppressants and obesity medications such as phenteramine or appetite-stimulating medications such as Megace, immunosuppresive agents, vasodilators like nitrates, BoTox and other therapeutic toxins and antitoxins, dyes and other markers. The hydrophilic polymer composition may be applied to one side of a two-sided patch. The other side can contain antibiotics, chemotherapeutic agents, minerals and vitamins, appetite-suppressants and obesity medications such as phenteramine or appetite-stimulating medications such as Megace, corticosteroids, immunosuppresive agents, vasodilators like nitrates, BoTox and other therapeutic toxins and antitoxins, corticosteroids, antihistamines, dyes and other markers.

The may be used to coat a device such as a mouth guard, tray for whitening teeth or taking teeth impressions. Typically these will be applied as a paste, gel or film to the device at the time of use.

The hydrophilic polymer composition can be incorporated into cosmetics or makeup, to reduce inflammation or alleviate pain at the same time as covering up the inflammation or painful site.

The hydrophilic polymer composition can be incorporated into or onto or in a kit with needles or catheters or ports. This may be particularly advantageous with tattoo needles or piercing jewelry. These may be in the form of wipes or sponges that are applied to the skin at the time of or immediately before application of the needle, or even added to the tattoo ink or applied as a coating to the needle.

### II. Methods of Treatment or Prevention

A. Methods of Administration

The composition is applied topically to a site at or adjacent to a painful region for both localized and systemic effects. The composition is reapplied as necessary. Pain relief is typically obtained within minutes and lasts for variable periods depending on the patient and type of pain symptoms. The compounds are applied such that the dosage is sufficient to provide an effective dose in the painful area or immediately adjacent areas, to ameliorate or eliminate one or more symptoms causing pain, or pain. The composition is applied to the skin, which may be rubbed in using an applicator, to the site of pain, as needed. Ultrasound or heat may also be applied to increase transdermal penetration and to increase local vasodilation.

As used herein, topical includes injection or infusion at the site of administration, for example, subcutaneously, and can include administration to mucosal surfaces, as well as trans-rectal, intra-peritoneal, intra-uterine and intra-articular.

B. Therapeutic Indications

The composition is generally effective to treat visceral, somatic and neuropathic pain both acute and chronic as well as muscle pain and stiffness and joint pain and stiffness. Examples include joint, muscle and tendon pain, joint, muscle and tendon immobility, inflammatory pain, neuropathies, muscle spasms, osteoarthritis, breathing disorders such as wheezing, hunger pains, some types of headaches, dysphagia, fibromyalgia, autoimmune disorders, and pancreatitis.

The composition also has an effect on some of the psychological and vegetative symptoms of pain, especially chronic pain, since in several patients, the hydrophilic material, alone or in combination with a lipophilic vehicle, without any active ingredient, applied to different areas on the skin can produce beneficial systemic effects such as decreased appetite, a feeling of heightened alertness, decongestion, increased energy and decreased fatigue, bronchodilation, urinary retention, and a sensation of decreased work of breathing. The composition has been demonstrated to provide pain relief in human patients for a wide number of conditions

Indications for which the present formulations can be used include, but are not limited to, inflammatory arthropathies including rheumatoid arthritis, lupus and Reiter's syndrome, neuropathies including those resulting from pressure, medication and diabetes, bursitis, tendinopathies, sprains and muscle strains, joint pains and arthralgias, muscle stiffness and overuse syndromes, pancreatitis, dyspnea, wheezing and chest tightness induced by asthmas, atelectasis, high blood pressure, obesity and chronic obstructive pulmonary disease (COPD), tension headaches, pain from anal fissures, hunger pain, fractures or compression of lumbar vertebrae, fibromyalgia, chronic coccygeal pain, reflex sympathetic dystrophy, polyneuropathy, TMJ dysfunction, and osteoarthritis/degenerative joint disease, spondylosis, sunburns, insect stings, and blisters.

The present invention will be further understood by reference to the following non-limiting examples. The examples demonstrate a significant decrease in the reported neuropathic pain, joint pain and stiffness, muscle pain and stiffness leading to increased mobility and range of motion of subjects receiving treatment of topically applied compounds as compared to subjects receiving placebo therapy. Subjects receiving treatment of topically applied compounds reported a rubifacient effect on the skin, ranging from mild to pronounced in some cases, as compared to subjects receiving placebo therapy. The first set of examples refers to treatment of patients with the compositions, usually in combination with an active agent. The second set of examples refers to treatment of patients with the compositions alone and with an emphasis on the systemic effects observed both after administration of the composition to the affected painful area and to different non-involved cutaneous areas.

### EXAMPLE 1

### Administration of 15 or 20% PLURONIC™, to Alleviate Pain

(a). PLURONIC™ 20% was Applied to Human Control.

PLURONIC™ 20% with 750 mg lactose (Weise compounding pharmacy) was applied to skin of normal control human patient, resulting in numbness similar to that produced by lidocaine.

(b) PLURONIC™ Gel 15% was Administered to a Patient with Pain from Occipital Neuritis.

Patient presented with 3/10 pain from occipital neuritis complained of local tenderness at the base of the scalp with headaches and paresthesias over the scalp. PLURONIC™ gel 15% was rubbed over the base of the skull with the result that pain levels dropped to 1.5/10.

(c) PLURONIC™ 15% Gel was Administered to a Patient with Pain from Tension Headache.

Patient presented with a chronic tension-type headache 7/10 in intensity manifested by a pressing, tightening quality bilaterally over the temples and back of neck. PLURONIC™ gel 15% was rubbed over the temples and back of neck with a dramatic relief of pain to 2/10.

(d) PLURONIC™ 15% Gel was Administered to a Patient with Pain from Biceps Tendonitis.

Patient with 4/10 pain from biceps tendinitis complained of shoulder pain aggravated by lifting and overhead reaching with local tenderness in the bicipital groove. PLURONIC™ gel 15% was rubbed over the bicipital groove and patient noted pain relief to 2/10.

(e) PLURONIC™ 15% Gel was Administered to a Patient with Pain Following Venipuneture.

Patient received venipuncture and had 2/10 stinging pain over the antecubital fossa. PLURONIC™ gel 15% was rubbed over the area and the pain and stinging disappeared almost immediately.

(f) Administration Following a Fingerstick
(i) Patient was given a fingerstick on right index finger without PLURONIC™ gel, which was painful; (blood sugar: 123 mg/dl), fingerstick on left index finger with PLURONIC™ gel 15% stick was less painful (blood sugar measured 132 mg/dl).
(ii) 15% PLURONIC™ was applied on the right index finger of a patient, and nothing was placed on the left index finger. A fingerstick was done on both fingers. He barely felt the needlestick on the right index finger, while on the left index finger he felt a sharp pain and discomfort which lingered even after the fingerstick. Applying 15% PLURONIC™ gel over this finger completely eliminated the discomfort. His glycemia measured on the left was 128 and on the right it was 114. When the blood was remeasured on other fingers without the gel, it was 109 on the left and 102 on the right.
(iii) Finger sticks were performed on 20 patients using the TheraSense device glucometer. Although this device requires the smallest drop of blood and produces the least discomfort, it still results in a noticeable pain which lasts about ten to twenty minutes. To perform the finger stick, finger was cleaned with and alcohol wipe, allowed to dry, then a stick was performed and the glucose concentration measured. Using the same finger PLURONIC™ gel 15% was applied, allowed to dry, and a stick was performed. The pain was so reduced as to be barely noticeable and the meter reading was within 10% of the initial stick. Of note if the is the fact that the specifications for these meters allows for 20% variation for the device.

### EXAMPLE 2

### Administration of PEG 300 mw Liquid in the Absence of Therapeutic Agents for Relief of Pain

(a) Administration to a Patient with Pain from Ulnar Entrapment

Patient presented with ulnar entrapment at the elbow complained of pain and tenderness at the elbow 8/10 radiating down the medial aspect of the right arm along with numbness and tingling in the 4th and 5th digits and loss of motor function manifesting as clumsiness, loss of dexterity and weakened grip. Claw hand with bent 4th and 5th fingers was present and Froment's sign was elicited. About 0.5 ml of PEG MW 300 was rubbed over the cubital tunnel and minutes later the patient reported that the paresthesias had disappeared and motor function had returned slightly with patient able to unbend the 4th and 5th fingers. Pain levels dropped to 3/10.

(b) Administration to a Patient with Pain from Rheumatoid Arthritis

Patient presented with rheumatoid arthritis complained of 7/10 joint pain the hands and ankles with swelling, tenderness, and limited motion. The inflamed joints of the hands had developed characteristic swan-neck and boutoimiere's deformities and arthritis in the forefoot ankles and subtalar joints produced severe pain with ambulation. About 0.5 ml of PEG MW 300 was rubbed onto the gums and placed under the tongue with the result that the patient noted more mobility in her hands and feet and pain levels improved, according to the patient, by about 50% to 3.5/10.

(c) Administration to a patient with Pain Following Motor Vehicle Accident

Patient presented after a motor vehicle accident with mild 3/10 cervical strain characterized by edema of cervical tissues with a palpable bogginess of the cervical posterior musculature, tightness, and increased cervical muscle tension, mild warmth over the neck, and limited cervical range of motion due to muscle spasm. About 0.5 ml of PEG MW 300 was rubbed over the gums and placed under the tongue and the patient stated that his neck mobility improved and pain levels dropped to about 1/10.

(d) Administration to a Patient with Pain from Sunburn

Patient presented with sunburn over the upper arms, back, neck and torso with 7/10 pain characterized by warmth, edema, tenderness and swelling. PEG MW 300 was rubbed onto these areas and the patient reported immediate relief with pain levels reducing to 3/10.

(e) Administration to a Patient with Pain from Subscapular Bursitis

Patient with subscapular bursitis characterized by localized tenderness under the superomedial angle of the scapula over the 2nd rib 5/10 in pain intensity. PEG MW 300 was rubbed over the area with the result that pain levels decreased only slightly to 4/10.

(f) Administration to a Patient with Pain from Sacroillitis

Patient with sacroillitis complained of severe 8/10 pain localized to the lumbosacral spine with occasional radiation into the gluteal muscles. About 0.5 ml of high molecular weight PEG was rubbed over the gums with the result that patient's pain levels dropped to 6/10.

(g) Administration to a Patient with Pain from a Periapical Abcess

Patient presented with 10/10 pain from a periapical abscess, About 0.5 ml of PEG MW 300 was rubbed over the affected area and the patient noted immediate numbness with complete resolution of the pain.

(h) Administration to a Patient with Wrist and Neck Pain

Male patient presented with wrist pain with a pain level of 7/10 and neck pain with a pain level of 5/10. Administration of PEG reduced his neck pain levels to 2/10.

### EXAMPLE 3

### Administration of Surgilube for Pain Relief

(a) Administration to a Patient with Pain and Weakness of the Forearm from Medial Epicondylitis

Patient presented with a 6/10 elbow pain, and weakness of the forearm from medial epicondylitis. Surgilube was applied to a dime-sized area just distal to the medial epicondyle and within 1 minute the patient reported that the pain completely disappeared.

(b) Administration to a Patient with Pain from De Quervain's Tenosynovitis

Patient presented with s 4/10 wrist pain and difficulty with gripping due to De Quervain's tenosynovitis. Surgilube was applied over the distal portion of the radial styloid adjacent to the abductor pollicis longus tendon and within seconds the pain disappeared.

(c) Administration to a Patient with Pain Due to Gamekeeper's Thumb

Patient presented with a 8/10 pain and swelling along the ulnar side of the metacarpophalangeal joint due to gamekeeper's thumb from an acute injury to ulnar collateral ligament of the thumb. Surgilube was applied to the ulnar side of the metacarpophalangeal joint and the pain decreased to 1/10.

(d) Administration to a Patient with Pain in Ringers

Patient presented with a loss of sensation in the tips of the 1st three fingers and 3/10 pain traveling through the wrist. Surgilube was applied over the wrist and within 2 minutes the patient reported that her pain levels and paresthesias completely disappeared.

(e) Administration to a Patient with Pain from Guillain-Barre Syndrome

Patient presented with Guillain-Barre syndrome complained of severe paresthesias and 10/10 pain in the legs and feet due to bilateral peroneal, tibial and sural neuropathies. Surgilube was applied over the fibular heads and adjacent to the lateral and medial malleolus with a dramatic reduction in pain levels to 3/10 and a general diminishment in paresthesias. The relief of symptoms lasted about 12 hours.

(f) Administration to a Patient with Pain from Venipuncture

Patient presented with a 1/10 pain from venipuncture. Surgilube was rubbed on the antecubital fossa of a patient following venipuncture. The discomfort which was described as 1/10 immediately disappeared.

(g) Administration to a Patient with Pain from a Needlestick

Patient underwent a diabetic needlestick in the right index finger. The pain level was 2/10 and the measured glycemia was 110 mg/dl. Surgilube was then applied to his left index finger and after 60 seconds had elapsed the finger was pricked with the lancet. He reported that although he still felt the needlestick the pain was less about 1/10 and the glycemia measured was 107 mg/dl, indicating the polymer did not significantly alter the glucose measurement.

(h) Administration to a Patient with Pain from Biceps Tendinitis

Patient presented with a 6/10 shoulder pain due to biceps tendinitis reported complete resolution of symptoms after Surgilube was rubbed over the bicipital groove approximately 1 inch below the anterolateral tip of the acromion.

(i) Administration to a Patient with Pain from Osteoarthritis

Patient presented with 8/10 bilateral knee pain, stiffness and crepitus due to osteoarthritis of the knees. Surgilube was rubbed over the knees and within seconds the patient reported that all pain had disappeared. She demonstrated the pain relief by leaping up and down repeatedly. This patient had been taking high doses of methadone and Lortab because of the pain. However, she had been given a large quantity of Surgilube to take home with her and, because she stated that the pain relief lasted at least 12 hours after each application, she has been able to cut back considerably on the amount of narcotics she was taking.

### EXAMPLE 4

### Administration of Povidone (Powdered USP 30 Povidone Mixed with a Few Milliliters of Bottled Water) Administered for Pain Relief

(a) Administration to a Patient with Pain from Guillain-Barre Syndrome

The same patient with Guillain-Barre syndrome as in example (e) under Administration of Surgilube returned 1 week later with complaints of severe paresthesias and 10/10 pain in the legs and feet. A solution of povidone USP powder semi-dissolved in water was rubbed over the affected areas as in example 5 with a similar reduction in pain levels and paresthesias. The relief of symptoms lasted at least 1 hour which was the length of time that the patient remained in the office after rubbing the povidone/water mixture on the affected areas.

(b) Administration to a Patient with Pain from Tendon Cyst

Patient presented with a tendon cyst complained of a 2/10 pain when the nodule was compressed. A solution of povidone semi-dissolved in water was rubbed over the nodule in the palm and the patient reported that he no longer felt pain when the nodule was compressed.

(c) Administration to a Patient with Pain from Costochondritis

Patient presented with a 8/10 pain in the anterior chest wall due to costochondritis with symptoms of extreme anxiety. The powdered povidone/water mixture was rubbed about 1 inch from the midline of the sternum and the patient reported that all pain disappeared along with his anxiety when he realized that he was not having a heart attack.

(d) Administration to a Patient with Lumbosacral Pain

Patient presented with a 8/10 lower back pain and muscle stiffness due to lumbosacral pain. Povidone/water mixture was applied topically about 2 inches away from the midline adjacent to L3-L4 over the maximum area of paraspinal muscle tenderness and spasm. After about 2 minutes the patient reported that his pain levels dropped to 2/10 and he was able to flex forward at the waist and bend to the side, two maneuvers which he had previously been unable to perform.

(e) Administration to a Patient with Pain from Lumbar Radiculopathy

Patient presented with a 7/10 back pain due to lumbar radiculopathy. Povidone/water solution was rubbed over the spinous processes but the patient reported no relief.

(f) Administration to a Patient with Pain from Sacroiliac Strain

Patient presented with a 6/10 pain and stiffness in the bottom of the lumbosacral spine with radiation down the leg due to sacroiliac strain. Povidone/water solution was rubbed over the left sacroiliac joint with complete resolution of the pain in the low back and leg and with improved flexibility.

(g) Administration to a Patient with Pain from Hyperesthesia after Spider Bite

Patient presented with severe bullae formation, cyanosis, and a 10/10 pain from hyperesthesia over the buttocks due to a bite from a brown recluse spider which was bandaged. The patient was in so much pain that he found it impossible to sit. Povidone/water solution was rubbed over the thin bandage and within 1 minute the patient reported extreme pain relief to 4/10 and, even though he still felt extreme discomfort, he was able to sit again on a chair.

(h) Administration to a Patient with Pain from Venipuncture

Povidone/water mixture was rubbed over a venipuncture site on a patient. The pain decreased from 2/10 to 0/10.

(i) Administration to a Patient with Pain from Cervical Strain

Patient presented with a 4/10 neck pain, stiffness and tightness from chronic cervical strain reported total relief after povidone/water mixture was rubbed over his neck.

(j) Administration to a Patient with Pain from Cervical Radiculopathy

Patient presented with 5/10 neck pain from cervical radiculopathy reported mild to no pain relief after povidone solution was rubbed over his neck.

(k) Administration to a Patient with Pain from Subscapular Bursitis

A patient with 5/10 pain from subscapular bursitis reported that his pain was reduced to 1/10 after povidone/water solution was rubbed under the superomedial angle of his left scapula over the second and third ribs

Modifications and variations will be obvious to those of skill in the art from the foregoing detailed description of the invention and are intended to come within the scope of the following claims.

We claim:

1. A method for the treatment of pain comprising topically administering to the skin at or adjacent to the site in need of treatment for pain, an effective amount of a formulation comprising a hydrophilic polymeric material effective in the absence of pharmaceutically active agents to alleviate one or more symptoms associated with the pain,
wherein the formulation is in the form of a cream, gel, lotion, spray, foam, paste, patch or pad, suspension or dispersion, for topical application to the skin or mucosal surface.

2. The method of claim 1, wherein the formulation comprises a polyalkylene polymer or copolymer.

3. The method of claim 1, wherein the formulation comprises a polyalkylene oxide block copolymer.

4. The method of claim 1 further comprising a penetration enhancer.

5. The method of claim 1 for producing systemic effects selected from the group consisting of decreased appetite, a feeling of heightened alertness, decongestion, increased energy and decreased fatigue, bronchodilation, urinary retention, and a sensation of decreased work of breathing.

6. The method of claim 1 comprising treating of pain arising from or associated with acute and chronic pain, which can be somatic, visceral, neuropathic, autoimmune, inflammatory, joint or muscle stiffness.

7. The method of claim 1 wherein the pain arises from or is associated with joint, muscle or tendon pain.

8. The method of claim 1 wherein the pain arises from or is associated with headache.

9. The method of claim 1 wherein the pain arises from or is associated with hunger.

10. The method of claim 1 wherein the pain arises from or is associated with dysphagia, fibromyalgia, or autoimmune disorders.

11. The method of claim 1 wherein the pain arises from or is associated with neuropathic pain.

12. The method of claim 1 wherein the pain arises from, or is associated with, difficulty in breathing or breathing spasms.

13. A formulation for the topical treatment of pain comprising an effective amount of a formulation of a hydrophilic polymeric excipient in an amount effective to alleviate one or more symptoms associated with the pain in the absence of pharmaceutically active agents, wherein the formulation is in the form of a cream, gel, lotion, spray, foam, paste, patch or pad, suspension or dispersion, for topical application to the skin or mucosal surface.

14. The formulation of claim 13 further comprising a disinfectant, preservative or antiseptic.

15. The formulation of claim 13 applied to the surface of or impregnated into bandages, gauze, adhesive strips, wipes, wraps, sponge, cotton swab, patch, glove, sock, wrist bands, fabric, fibers, sutures, medication pad, underwear, tissue, pain-relief gel pack or bedliner, lip balm, poultice, plaster, or compress.

16. The formulation of claim 13 applied to or impregnated in adult diapers, pads, guards or liners to promote continence.

17. The formulation of claim 13 applied to or impregnated into a patch, bandage or dressing around the neck to decrease obstructive sleep apnea.

18. The formulation of claim 13 in a kit comprising a sealed sterile amount of the formulation for administration with a device to an individual in need thereof.

19. The formulation of claim 10 applied to, or administered with, needles, catheters, tubing, tubing or needle ports, mouth guard, or dental tray.

20. The formulation of claim 10 applied to or administered with jewelry or tattoo needles.

21. The formulation of claim 10 formulated with cosmetics for application to the skin for relief of pain.
